(19) United States
(12) Patent Application Publication
(10) Pub. No.: US 2015/0017717 A1
(43) Pub. Date: Jan. 15, 2015

(54) CULTURE MEDIUM FOR EUKARYOTIC CELLS

(71) Applicant: Friesland Brands B.V., Amersfoort (NL)

(72) Inventors: Abhishek Gupta, Wageningen (NL); Mireille Maria Gadellaa, Bemmel (NL); Dominick Yves Willy Maes, Ede (NL)

(73) Assignee: Friesland Brands B.V., Amersfoort (NL)

(21) Appl. No.: 14/383,534

(22) PCT Filed: Mar. 8, 2013
(86) PCT No.: PCT/NL2013/050154

§ 371 Date: Sep. 5, 2014

(30) Foreign Application Priority Data
Mar. 8, 2012 (EP) 12158585.5
Mar. 8, 2012 (EP) 12158586.3
Mar. 8, 2012 (EP) 12158598.8
Mar. 8, 2012 (EP) 12158604.4
Mar. 8, 2012 (EP) 12158607.7

(51) Int. Cl. C12N 5/00

(52) U.S. Cl. CPC C12N 5/0043; C12N 2501/999; C12N 2501/40; C12N 2500/92; C12N 2500/32
USPC 435/358; 435/431

(57) ABSTRACT

The invention pertains to the use of C₂-C₆alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid and combinations thereof, as a growth- and production promoting ingredient, in culture media for culturing eukaryotic cells. The invention further pertains to culture media containing these alpha-hydroxy acid derivatives at levels of at least 0.001 mg/l.

### FIELD OF THE INVENTION

The invention relates to the production of a medium for culturing eukaryotic, in particular animal cells, as well as to a cell culture medium thus produced and its use for in vitro cultivation of eukaryotic, in particular animals cells.

### BACKGROUND

The production of valuable biochemicals and biopharmaceuticals, for instance antibodies and antibiotics, by culturing mammalian, plant or insect cells requires proper culture media. Cell culture media formulations have been supplemented with a range of additives, including undefined components like fetal calf serum (FCS), several animal-derived proteins and/or protein hydrolysates of bovine origin.

Serum or serum-derived substances, such as albumin, transferrin or insulin, which are used in animal cell culture, may contain unwanted agents that can contaminate the cultures and the biopharmaceutical products obtained from these. Moreover, bovine derived protein products like bovine meat or collagen hydrolysates bear the risk of BSE contamination. Furthermore, additives derived from human serum have to be tested for all known viruses, including hepatitis and HIV that can be transmitted by serum.

In conclusion, all serum-derived products can be contaminated by unknown agents. In the case of serum or protein additives that are derived from human or other animal sources in cell culture, numerous problems (e.g. the varying quality and composition of different batches and the risk of contamination with viruses, mycoplasma or BSE) can occur. Therefore, plant protein hydrolysates or plant peptones are commonly used in culture media that should be free of animal components.

However, growth and productivity of animal cells in media without animal-derived cell culture additives is not always satisfactory. It is frequently observed that animal cells which are cultivated in vitro grow in lumps. This is considered to be a suboptimal condition as the cells in the core of the lump are deprived of nutrients and will die. There is also a risk of clogging the tubing or the filters during downstream processing. The reduced viability of the cells can also be assessed by their appearance. Cells having a reduced viability show an irregular shape, i.e. a not-round shape, and in addition have a “granulated” cell content which is in contrast to healthy cells that have perfectly bright and transparent cell content.

WO 2006/123926 relates to a peptide composition for growing and/or culturing micro-organisms and/or cells on the basis of at least one vegetable protein source, preferably from rapeseed, wheat or caraway. The effect of wheat hydrolysate is addressed in the examples.

WO 2006/128764 discloses a process for cultivating mammalian cells producing complex proteins, wherein one or more plant-derived peptones are fed to the cell culture. Plant sources soy, cotton seed and pea are exemplified. The effect of soybean hydrolysate on cultivation of CHO cells is shown in the accompanying examples.

WO 2009/020389 discloses the use of a protein hydrolysate of Helianthus (sunflower) species as a constituent of a culture medium for culturing eukaryotic, in particular animal cells.

US2003/0203448A1 describes a protein-free and serum-free medium for the cultivation of cells, comprising soy hydrolysate and optionally added free amino acids.

Nicoll et al., Connective Tissue Research, vol. 42, no. 1, pp. 59-69, disclose a study of the effect of lactic acid on the induction of chondrogenesis (cartilage development) in human dermal fibroblasts. Thereto, cells were grown in a culture medium comprising minimal essential medium (MEM) supplemented with 10% fetal bovine serum (FBS), penicillin, streptomycin and 80 mM lactic acid.

Morishima et al., Neuroscience Research, vol. 61, no. 1, pp. 18-26, study the effect of lactic acid on the expression and localization of the water channel protein aquaporin (AQP4) in cultured rat astrocytes. To this effect, astrocyte cells were cultured in a medium comprising Dulbecco's Modified Eagle's Medium (DMEM), 10% fetal bovine serum (FBS) and 15-45 mM lactic acid or lactate.

Lampe et al., Biotechnology and Bioengineering, vol. 103, no. 6, pp. 1214-1223, disclose an investigation of the effects of lactic acid on neural precursor cells. Thereto, cells are cultured in a medium containing DMEM, penicillin, streptomycin, N2, L-glutamine, basic fibroblast growth factor (bFGF) and up to 10 mg/ml of lactic acid.

Kromenaker et al., J. Biotechnol., vol. 34, 1994, pages 13-34, describe the effects of lactic acid concentration on the cell cycle kinetics of hybridoma cell growth and antibody production. Cells were cultured in a medium comprising DMEM, penicillin, streptomycin, heat-inactivated horse serum and up to 140 mM of lactic acid.

Ballez et al. discloses a protein-free medium for cell cultivation, comprising a basal defined medium (BDM) supplemented with 0.1% pluronic F68, 0.04 pM sodium selenite and 500 μM ferric citrate (p. 105, top), as well as rice or wheat protein hydrolysate.

US2002/0039787 discloses a method for the in vitro culturing of microvascular endothelial cells, said method comprising culturing an enriched population of microvascular endothelial cells in the presence of an effective amount of human serum.

The functionality of the plant protein hydrolysates is a direct result of its chemical composition. It is affected by several factors like raw material, processing factors, process control and storage conditions. Therefore, it results in a persistent yet poorly studied phenomenon defined as “lot-to-lot variation”.

It is a major concern expressed by the biopharma industries, which is the customer of these hydrolysates, as it can mean variations in the product yields from 10 to 25% and it has direct financial consequences. The invention aims at relieving these concerns.

### SUMMARY OF THE INVENTION

It was found that specific C₂-C₆ alpha-hydroxy acids, salts of these acids, esters of these acids and combinations thereof have a strong growth and production promoting effect on cell cultures of eukaryotic cells, especially animal cells in vitro. The presence of a minimum level of these compounds results in consistent and therefore commercially attractive production performance. Media containing these derivatives are excellently suitable for culturing eukaryotic, in particular animal cells. Thus the invention provides a cell culture medium containing such specific alpha-hydroxy acids, their salts or their esters, as well as a process of producing these media and a method for cultivation of animal cells in vitro using compositions containing these alpha-hydroxy acids, their salts or their esters as a medium constituent.

### DETAILED DESCRIPTION OF THE INVENTION

The invention pertains to a process of producing a culture medium for culturing eukaryotic cells, in particular animal cells, involving the use of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids and combinations thereof, as a growth-promoting or production-improving ingredient. The present invention also pertains to a medium for culturing eukaryotic, in particular animal cells, containing at least at least 0.02 ppm (0.02 mg/kg), preferably at least 0.2 mg/kg, more preferably at least 2 mg/kg, even more preferably at least 20 mg/kg, most preferably at least 50 ppm (50 mg/kg), on a dry weight basis, of one or more of the above alpha-hydroxy acids or their derivatives.

Wherever in the present description amounts of ingredients of the cell culture medium of the invention are given on a dry weight basis, the final concentrations in the liquid medium can be derived by arbitrarily taking a dry solids content of 5% (50 g/l) and vice versa. Thus, an amount of 100 mg per kg of dry matter, corresponds, for the sake of deriving preferred levels, to 5 mg per l of the final liquid medium. This by no means implies that the dry solids content of the liquid medium should be 5%. Depending on the specific cell culture concentrations, dry solid levels of e.g. between 0.5 and 30 wt. %, preferably between 0.5 and 15 wt. %, more preferably between 1 and 15 wt. %, most preferably between 1 and 5 wt % can be chosen.

The C₂-C₆ alpha-hydroxy acids for use according to the present invention may optionally be substituted at the C₂-C₆ backbone. Suitable substituents include halogen, ester, ether, hydroxyl, amino and aromatic groups.

Preferred salts are the sodium, potassium, calcium, magnesium or ammonium salts of the C₂-C₆ alpha-hydroxy acids of the invention.

Preferred esters are the linear or branched C₁-C₄ esters of the C₂-C₆ alpha-hydroxy acids of the invention.

Particularly preferred compounds for use according to the invention are mucic acid and methyl-L-3-phenyl lactate.

The alpha-hydroxy acids, their salts and their esters to be used according to the invention can be used as such. Most of the components are commercially available. Alternatively, they can be produced by commonly known synthetic or semi-synthetic procedures. Most of the derivatives can also be isolated from suitable protein fractions or hydrolysates, especially plant-derived proteins such as from soybeans, peas, lentils, wheat (gluten), cottonseed, rice, sunflower, safflower etc. They can be extracted or enriched from the protein fraction, or more conveniently from protein hydrolysates. Such methods are known in the art.

The invention thus concerns a process of producing a cell culture medium by adding to further constituents of the medium an amount of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids, and combinations thereof, such that the final concentration in the medium is at least 0.001 mg/l, preferably at least 0.01 mg/l, more preferably at least 0.1 mg/l, most preferably at least 1 mg/l per individual alpha-hydroxy acid or derivative according to the invention, and as further elaborated below. It is preferred that the final concentration in the medium is at most 50 g/l, preferably at most 1 g/l, more preferably at most 100 mg/l per individual alpha-hydroxy acid, salt of this acid, or ester of this acid. The derivatives can be added as such, e.g. as purified and/or synthetic products. Preferably, such purified and/or synthetic components have a purity of at least 80%, more preferably at least 90%, most preferably at least 95%. Alternatively, the compounds can be added as a concentrate, i.e. a product obtainable by concentrating or enriching a solution of one or more alpha-hydroxy acids or derivatives thereof according to the invention to a level of at least 0.01% by weight, preferably at least 0.1%, more preferably at least 1%, most preferably at least 10%, or even at least 25% and preferably at most 80%, more preferably at most 70% by weight, or having a concentration of preferably at least 0.1 mg, more preferably at least 1 mg, even more preferably at least 10 mg, even more preferably at least 1 g, yet even more preferably at least 10 g, most preferably at least 100 g per kg of dry matter.

Within the context of the present invention, the terms “further constituents of the medium” and “further conventional medium ingredients” refers to compounds that are commonly known in the art as constituents of cell culture media, such as plant or animal cytokines and/or growth factors (provided that these are not of animal origin), vitamins, minerals, amino acids, buffering salts, trace elements, nucleosides, nucleotides, phytohormones, sugars including glucose, antibiotics and the like. Phytohormones comprise auxins, gibberellins, abscisic acid and combinations thereof. Depending on the cell line and cell aspects envisaged, the skilled person will be able to select the types and quantities of further medium constituents desired or required.

The invention further pertains to a cell culture medium obtainable by this process. More specifically, the invention relates to a culture medium for culturing eukaryotic cells containing at least 0.001 mg per l, preferably at least 0.01 mg per l, more preferably at least 0.1 mg per l, even more preferably at least 1 mg per l, most preferably at least 5 mg per l of final liquid medium of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids, or combinations thereof, wherein the concentrations are per individual component. It is preferred that the final concentration in the medium is at most 50 g/l, preferably at most 1 g/l, more preferably at most 100 mg/l per said individual component. In terms of dry weight of the cell culture medium of the invention, it contains at least 0.02 mg per kg, preferably at least 0.2 mg per kg, more preferably at least 2 mg per kg, even more preferably at least 20 mg per kg, most preferably at least 250 mg per kg of dry matter, and at most 1000 g, preferably at most 20 g, more preferably at most 2 g per kg of dry matter of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids or combinations thereof, wherein the concentrations are per individual component.

In a preferred embodiment of the invention, a cell culture medium contains one or more of the above alpha-hydroxy acids or their esters or salts in a concentration of between 5 mg/l and 30 g/l per individual component, or between 100 mg and 600 g, preferably between 250 mg and 150 g per kg dry matter. More preferred levels are between 10 mg/l and 1 g/l or between 200 mg and 100 g, preferably between 500 mg and 50 g per kg dry matter, even more preferred between 20 mg/l and 500 mg/l or between 1 and 25 g per kg dry matter.

In one embodiment of the invention, the alpha-hydroxy acids, their salts or their esters according to the invention are used as part of one or more plant protein hydrolysates.

In a particularly preferred embodiment of the invention, the one or more alpha-hydroxy acids, their salts or their esters, or combinations thereof, are used in combination with or added to one or more plant protein hydrolysates.

The amount of (essentially water-soluble) hydrolysate in the liquid medium can be determined by the skilled person, but comprises preferably 0.001-10.0 wt/vol %, more preferably 0.01-10.0 wt/vol %, more preferably 0.01-4.0 wt/vol %, even more preferably 0.05-2.0 wt/vol %, or 0.05-1.0 wt/vol %, even more preferably 0.1-1.0 wt/vol %, and most preferably 0.2-0.6 wt/vol %.

The protein hydrolysates can be produced by methods known in the art, e.g. by processing the beans, legumes, seeds etc. by pressing, grinding, dehulling and/or crushing, if desired followed by defatting, e.g. using organic solvents such as hexane. Preferably the defatted seed material contains at least 20 wt % protein. The defatted seed material preferably has a fat content of less than 10 wt. %.

A protein hydrolysate is usually obtained by enzymatic proteolysis and can also be referred to as proteolysate. The (defatted) plant seed material, optionally comminuted, is subjected to hydrolysis using endo and/or exo proteases from bacterial, fungal, vegetable or animal origin or mixtures thereof; however preferably the enzyme is not from an animal source. The enzyme may be produced using recombinant DNA techniques. The preferred enzymes are endo-proteases. More preferably the enzyme comprises alkaline proteases. Suitable proteases include a subtilisin (Alcalase), a serine endoprotease. Particularly suitable enzymes comprise Alcalase from Novozymes, and/or papain from Merck. Other suitable enzymes comprise e.g. Neutrase.

Hydrolysis conditions comprise a reaction time of between 30 minutes and 30 hours; preferably 1-6 hours, most preferably 2-4 hours; temperatures are between 20 and 65° C., preferably between 40° C. and 60° C., all depending on the particular protein source and the desired degree of hydrolysis. The pH may be adjusted between 6.0 and 8.5, preferably 6.6 and 8.0, most preferred is 7.0-8.0. The concentration of the protein to be hydrolysed in solution is between 1 and 10% protein, preferably 2-8, most preferably 3-6 wt. %. The amount of enzyme used is, based on substrate, between 0.5-10 wt %, preferably 1-5 wt %, most preferably 1.5-3.5 wt %.

The hydrolysis is preferably performed until a degree of hydrolysis of between 5 and 50%, preferably between 10 and 40%, most preferably between 10 and 30%, is attained. The hydrolysis reaction is terminated using a heat treatment. Preferably, the heat treatment encompasses a heating time of between 15 and 90 minutes between 80 and 100° C. (batch heat treatment), or 1-5 minutes at 100-120° C. Degree of hydrolysis may be determined using conventional formol titration, as demonstrated in the examples. After termination of the hydrolysis reaction, the reaction mixture can optionally be polished to remove insoluble parts, for example using centrifugation or filtering aids know in the art like diatomaceous earth (e.g. Celite®, Dicalite®, Hyflo®). Preferably, the hydrolysate contains less than 10 wt. %, on dry matter basis, of water-insoluble material, more preferably less than 5 wt. %, most preferably less than 2 wt. %. The hydrolysate can be dried, for instance by spray drying or freeze drying. The hydrolysate may be used as such or may be further fractionated.

The hydrolysate preferably contains between 20 and 80 wt. %, especially between 20 and 60 wt. % of peptides having a molecular weight of 100-500 Da and/or between 10 and 30 wt. % of peptides of a molecular weight between 500 an 1000 Da on total protein basis. In terms of peptide length, the hydrolysate preferably contains at least 15 wt. %, more preferably at least 25 wt. %, most preferably at least 35 wt. %, up to e.g. 85 wt. %, more preferably up to 65 wt. %, most preferably up to 55 wt. % of di- to penta-peptides, between 8 and 30 wt. % of hexa- to nonapeptides, at least 8 wt. %, especially between 15 and 60 wt. % of higher peptides and between 0.1 and 30 wt. %, preferably between 0.5 and 10 wt. % of free amino acids, on total protein basis. In a preferred embodiment, the hydrolysate may be ultrafiltered, preferably using a 5 or 10 kDa molecular weight cut-off. The hydrolysate may contain further constituents such as carbohydrates, soluble fibres, multivalent metal salts, etc. Preferably the protein content (all proteinaceous material including free amino acids) is between 30 and 90 wt. %, more preferably between 45 and 85 wt. %. These amounts are on a dry weight basis.

The hydrolysate may be combined with other conventional constituents of culture media such as plant or animal cytokines and/or growth factors (provided that these are not of animal origin), vitamins, minerals, amino acids, buffering salts, trace elements, nucleosides, nucleotides, phytohormones, sugars including glucose, antibiotics and the like. Phytohormones comprise auxins, gibberellins, abscisic acid and combinations thereof.

The cell culture medium according to the invention may further contain one or more compounds selected from amino acid derivatives; phenolic acid derivatives; linear C₂-C₆ or cyclic C₆ sugar alcohols; pyridinic acid derivatives; and nucleobases and/or nucleotides chosen from uracil, adenine, adenosine 3′-monophosphate (3′-AMP) and adenosine 5′-monophosphate (AMP); and combinations of compounds from these compound groups. The concentration of these compounds is at least 0.001 mg per l, preferably at least 0.01 mg per l, more preferably at least 0.1 mg per l, even more preferably at least 1 mg per l, most preferably at least 5 mg per l of final liquid medium, wherein the concentrations are per individual component. It is preferred that the final concentration in the medium is at most 50 g/l, preferably at most 1 g/l, more preferably at most 100 mg/l per said individual component.

In a preferred embodiment, the cell culture medium further comprises one or more amino acid derivatives selected from γ-glutamyl amino acids, pyroglutamyl amino acids, glutamate-containing or proline-containing dipeptides, oxo-aminoacids, homo-amino acids, N-acetyl-amino-acids and glycyl-glycine. In a particularly preferred embodiment, the cell culture medium further comprises one or more amino acid derivatives selected from N-acetyl-methionine, N-acetyl-phenylalanine, N-acetyl-ornithine, γ-glutamyl-tyrosine, γ-glutamyl-phenylalanine, pyroglutamyl-glutamine, pyroglutamyl-glycine, valinyl-glutamate, glycylproline, cyclo-glycyl-glutamine, 5-oxoproline and S-oxo-methionine, and β-alanine, 2-aminobutyrate, glycyl-glycine and homoserine.

In another preferred embodiment, the cell culture medium further comprises one ore more phenolic acid derivatives, esters of these phenolic acid derivatives or salts of these phenolic acid derivatives. The term “phenolic acid derivatives” is understood to comprise all organic compounds having a C₁-C₆ skeleton that contain a phenolic ring and an organic carboxylic acid function, and which may further be substituted at the C₁-C₆ backbone and/or the phenolic ring, with e.g. halogen, ester, ether, hydroxyl, amino and/or aromatic groups. In a particularly preferred embodiment, the cell culture medium further comprises one or more phenolic acid derivatives selected from ferulic acid ((E)-3-(4-hydroxy-3-methoxy-phenyl)prop-2-enoic acid), syringic acid (4-hydroxy-3,5-dimethoxybenzoic acid), vanillic acid (4-hydroxy-3-methoxybenzoic acid), sinapinic acid (3-(4-hydroxy-3,5-dimethoxyphenyl)prop-2-enoic acid), C₁-C₄ esters of these acids, or salts of these acids. Most preferred phenolic acid derivatives are ferulic acid, syringic acid, C₁-C₄ esters of these acids, or salts of these acids.

In another preferred embodiment, the cell culture medium further comprises one ore more pyridinic acid derivatives. The term “pyridinic acid derivatives” is understood to comprise all organic compounds having a C₁-C₆ skeleton that contain a pyridinic (C₅H₄N) ring and an organic carboxylic acid function, and which may further be substituted at the C₁-C₆ backbone and/or the pyridinic ring, with e.g. halogen, ester, ether, hydroxyl, amino and/or aromatic groups. The pyridinic nitrogen may also be quaternized, i.e. alkylated with a linear, branched, saturated or unsaturated aliphatic or aromatic hydrocarbon.

Preferred salts are the sodium, potassium, calcium, magnesium or ammonium salts of the pyridinic acid derivatives of the invention.

Preferred esters are the linear or branched C₁-C₄ esters of the pyridinic acid derivatives of the invention.

A particularly preferred pyridinic acid derivative for use according to the present invention is trigonelline (1-methylpyridinium-3-carboxylate).

In another preferred embodiment, the cell culture medium further comprises one ore more linear C₂-C₆ sugar alcohols or cyclic C₆ sugar alcohols. Within the context of the present invention, the term “C₂-C₆ sugar alcohol” refers to linear compounds having the general formula H(HCHO)ₙ₊₁H, wherein n is 1, 3, 4, or 5, as well as to the cyclic C₆ compound cyclohexane-1,2,3,4,5,6-hexol. Typical examples of these compounds are xylitol, threitol, mannitol, and myo-inositol, allo-inositol, L-chiro-inositol and D-chiro-inositol, respectively. In a particularly preferred embodiment, the cell culture medium further comprises one or more sugar alcohols selected from chiro-inositol, erythritol, threitol and sorbitol, most preferably chiro-inositol.

In yet another preferred embodiment, the cell culture medium further comprises one or more nucleobases and/or nucleotides selected from uracil, adenine, adenosine 3′-monophosphate (3′-AMP) and adenosine 5′-monophosphate (AMP).

In a typical embodiment of the invention, the cell culture medium comprises one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids; and one or more amino acid derivatives selected from γ-glutamyl amino acids, pyroglutamyl amino acids, glutamate-containing or proline-containing dipeptides, oxo-aminoacids, homo-amino acids, N-acetyl-amino-acids and glycyl-glycine, wherein the concentration of these compounds is at least 0.001 mg per l, preferably at least 0.01 mg per l, more preferably at least 0.1 mg per l, even more preferably at least 1 mg per l, most preferably at least 5 mg per l of final liquid cell culture medium, wherein the concentrations are per individual component. It is preferred that the final concentration in the medium is at most 50 g/l, preferably at most 1 g/l, more preferably at most 100 mg/1 per said individual component

In another embodiment of the invention, the cell culture medium comprises one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids; one or more amino acid derivatives selected from γ-glutamyl amino acids, pyroglutamyl amino acids, glutamate-containing or proline-containing dipeptides, oxo-aminoacids, homo-amino acids, N-acetyl-amino-acids and glycyl-glycine; one ore more phenolic acid derivatives, esters of these phenolic acid derivatives or salts of these phenolic acid derivatives; one ore more linear C₂-C₆ sugar alcohols or cyclic C₆ sugar alcohols; one or more nucleobases and/or nucleotides selected from uracil, adenine, adenosine 3′-monophosphate (3′-AMP) and adenosine 5′-monophosphate (AMP); and trigonelline (1-methylpyridinium-3-carboxylate), wherein the concentration of these compounds is at least 0.001 mg per l, preferably at least 0.01 mg per l, more preferably at least 0.1 mg per l, even more preferably at least 1 mg per l, most preferably at least 5 mg per l of final liquid cell culture medium, wherein the concentrations are per individual component. It is preferred that the final concentration in the medium is at most 50 g/l, preferably at most 1 g/l, more preferably at most 100 mg/l per said individual component

Also commercially available basal media may be used in combination with the alpha-hydroxy acids of the invention and optionally the protein hydrolysates. For an animal cell line as CHO-1, CD-CHO, PowerCHO from Lonza, ISCHO-CD from Irvine Scientific, or Excell 325 PF CHO from SAFC may be used. For plant cells, Murashige and Skoog basal medium obtainable from SAFC may be used. The hydrolysate may also be a hydrolysate from different protein sources, such as hydrolysates from wheat and soy, soy and pea, rice and cottonseed. The cell culture medium preferably does not contain serum such as fetal calf serum, or serum-derived components in order to be full reproducible and/or to avoid contamination. Preferably, the cell-culture medium is free of animal components, such as animal-derived proteins and/or protein hydrolysates of animal, e.g. bovine, origin. Accordingly, in a preferred embodiment the invention pertains to a serum-free culture medium for culturing eukaryotic cells as defined herein, and to a process of preparing such a serum-free culture medium.

The cell culture medium and the method of culturing both according to the invention are capable of supporting cultivation of eukaryotic, in particular animal cells, where capability means that it enables at least the survival, proliferation and/or differentiation of—and preferably also the expression of product by the cells in vitro. Cultivation in batch, fed batch, continuous or perfusion reactors are all envisaged.

Cell growth curves can be separated in a real growth phase in which the cells multiply and grow, and a production phase, in which the cells are more or less in a steady state, but start to produce the metabolites of interest, e.g. antibodies. The alpha-hydroxy acids of the invention are capable of supporting both the growth phase and the production phase of animal or other eukaryotic cells.

The cell culture medium may be provided as a liquid or in a powdered, dried form. The amount of (essentially water-soluble) powdered or dried cell culture constituents in the liquid medium can be determined by the skilled person, but comprises preferably 0.01-10.0 wt/vol %, more preferably 0.01-4.0 wt/vol %, even more preferably 0.05-2.0 wt/vol %, or 0.05-1.0 wt/vol %, even more preferably 0.1-1.0 wt/vol %, and most preferably 0.2-0.6 wt/vol %.

The amount of hydrolysate in a dry culture medium that can be reconstituted with water is depending on the medium components, but is typically in the range of 2-80% w/w, preferably 5-50% w/w. The cell culture medium also preferably contains sugars, in particular glucose, preferably in a dry weight ratio of glucose to hydrolysate between 10 and 0.1, more preferably between 2.5 and 0.4, and further constituents as described above.

Furthermore, the invention concerns the use of the cell medium for culturing eukaryotic cells. Eukaryotes comprise Fungi (including yeasts), Protista, Chromista, Plantae and Metazoa (animals). The invention especially concerns the use for culturing plant cells, for example rice, tobacco and maize, and in particular animal cells, preferably in vitro cultivation. The cells to be cultured may be from a natural source or may be genetically modified. Animal cells especially comprise vertebrate and invertebrate cells, including mammalian cells such as human cells e.g. PER C6 cells®, rodent cells, in particular Chinese Hamster Ovary (CHO) cells, avian, fish, reptile, amphibian or insect cells.

The cells cultured by the method of the invention are in particular used for expression of protein products that may be further purified in biopharmaceutical industry. Non-limiting examples of protein products that can advantageously be produced in the culture medium of the invention include erythropoietin (for treating blood disorders), etanercept (TNF-α inhibitor for treating rheumatic diseases and gout), alpha dornase (deoxyribonuclease for the treatment of cystic fibrosis), beta-interferon (for treating multiple sclerosis) and a wide range of therapeutic monoclonal antibodies. The desired protein products may be recovered by methods known in the art, such as separating the cells from the culture medium and isolating the protein products from the cell-free liquid (supernatant) e.g. by fractionation, affinity chromatography (adsorption-desorption) or the like, or combinations thereof.

Furthermore, the invention concerns a kit comprising a fraction containing the one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids and combinations thereof, as well as one or more constituents of culture media selected from plant hydrolysates, plant or animal cytokines and/or growth factors, vitamins, minerals, amino acids, buffering salts, trace elements, nucleosides, phytohormones, nucleotides, sugars and antibiotics. The constituents may be present in the kit as one or more combinations. For example, the alpha-hydroxy acids of the invention may be separately present in dry or dissolved form and part or all of the further constituents of culture media such as plant hydrolysates, plant or animal cytokines and/or growth factors, vitamins, minerals, amino acids, buffering salts, trace elements, nucleosides, nucleotides, phytohormones, sugars and antibiotics, may be present as a separate combination. Alternatively, the alpha-hydroxy acids may be premixed with e.g. amino acids and/or peptides and/or sugars, and any remaining constituents may be present separately or in one or more combinations. It is preferred that at least one of the compositions is a liquid, which liquid may advantageously be sterilised. The compositions of the kit are mixed prior to use of the culture medium.

It has thus been found that the specific C₂-C₆ alpha-hydroxy acids, salts of these acids, esters of these acids according to the invention and their use have several important advantages. They have a growth promoting effect which exceeds the growth provided by common protein constituents. They result in enhanced production, a lower variance of production and/or growth, and are cost-effective.

Animal cells that are cultured in vitro are not growing in lumps or clusters but are present as single cells. Secondly, the viability of the cells is excellent as judged by their perfect round shape and bright transparent cell content. Thirdly, much higher cell densities can be obtained compared to state of the art cell culture media such as those based on non-serum protein, in particular soy protein, without compromising the expression level of the desired cell products. Fourthly, the C₂-C₆ alpha-hydroxy acids, their esters and their salts according to the present invention can be combined with any basal culture medium for in vitro cultivation of animal cells, enabling the manufacture of a wide variety of cell culture media with the advantages mentioned above. Also the cultivation can be extended over prolonged periods, resulting in higher product yields.

### EXAMPLES

### Example 1

### Analysis of Protein Hydrolysates Containing Claimed Alpha-Hydroxy Acids, Esters of These Acids, and Salts of These Acids, and Evidence of Growth Stimulation

Commercial plant protein hydrolysates like SE50MAF-UF, WGE80M-UF, CNE80M-UF, PCE80B obtained from FrieslandCampina Domo, USA were analysed by Liquid chromatography/Mass Spectrometry (LC/MS, LC/MS2) using a Waters Acquity UPLC and a Thermo-Finnigan LTQ mass spectrometer, which consists of an electrospray ionization (ESI) source and linear ion-trap (LIT) mass analyzer. The sample extract was split into two aliquots, dried, then reconstituted in acidic or basic LC-compatible solvents. One aliquot was analyzed using acidic positive ion optimized conditions and the other using basic negative ion optimized conditions in two independent injections using separate dedicated columns. Extracts reconstituted in acidic conditions were gradient eluted using water and methanol both containing 0.1% formic acid, while the basic extracts, which also use water/methanol, contain 6.5 mM ammonium bicarbonate. The MS analysis alternated between MS and data-dependent MS₂ scans using dynamic exclusion. Biochemicals were identified by comparison to metabolomic library entries of purified standards or recurrent unknown entities. The combination of chromato-graphic properties and mass spectra gives an indication of a match to the specific compound or an isobaric entity. Thus, an overview of the biochemical components and their relative concentration present in plant protein hydrolysates was generated. Furthermore, all the hydrolysates were tested in the cell culture assays for cell growth and antibody production. Linear regression analysis was performed on the cell growth and compound analysis data in order to identify the biochemical components that significantly affected the cell growth and antibody production. Using the SLOPE function of Microsoft Excel 2003, the correlation between antibody production and relative concentration of the components was calculated, the results of which are presented in Table 1 below. The higher the positive slope, the higher the importance of a biochemical component in the cell culture. p-values, also calculated using MS Excel's correlation regression function, represent the significance of the values, with the lower the p-value (all between 0 and 1), the more significant the measured value.

TABLE 1
Correlation of antibody production and relative
concentration of alpha-hydroxy acid derivates

| Biochemical component | SLOPE | p-value |
|---|---|---|
| mucic acid | 2108.11 | 0.0001 |
| alpha-hydroxyisovaleric acid | 2830.96 | 0.0001 |
| alpha-hydroxyisocaproic acid | 1654.23 | 0.0001 |

### Example 2

### Preparation of Cell Culture Medium

The cell culture assay was carried out in commercially available IS CHO-CD medium (Irvine Scientific, Cat. No. 91119). To this media, L-Glutamine (2 mM), pluronic acid, hypoxanthine (100 μM) and thymidine (15 μM) were added. Penicillin and streptomycin were added to prevent any bacterial growth during the growth assay. The media was supplemented with methyl-L-3-phenyl lactate or mucic acid, both purchased from Sigma Aldrich, Germany, in varying concentrations (1×10⁻³ to 1×10⁻¹% (w/v), see Table 2). The supplemented medium was mixed with a vortex mixture, filtered using a 0.22 μm filter and subsequently used in a growth assay.

### Example 3

### IgG Production and Cell Growth: In vitro Cultivation of CHO Cells

Cell Lines

An IgG expressing CHO cell line was used (CHO-2: ATCC CRL 11397, producing IgG4). The cell lines were grown in the adherent conditions for a few passages and once confluent, they were transferred to animal-free conditions in the supplemented media described in Example 2.

Growth and Production Curves

To measure growth and production curves, Chinese hamster ovary (CHO) cells were grown in suspension culture in baffled flasks. 20×10⁶ cells were transferred in 25 ml media to the baffled flasks. Chemically defined media with and without added alpha-hydroxy acid derivatives were tested. No fresh media was added during the growth assay. Cells were counted using the CEDEX HiRes cell counter (Innovatis, Germany). The cell counts were used to calculate the area under the growth curve and represented as dimensionless area under curve (AUC) values as described in detail in Ling, C. X, Huang, J. and Zhang, H. (2003), International joint conferences on artificial intelligence, pp. 329-341. The supernatant samples were taken every alternate days for the IgG production measurements. IgG production was measured using sandwich ELISA method. The specific IgG production was calculated by taking the ratio of cumulative IgG production (in mg/ml) and AUC measured at day 11 of the growth assay. The cells were visually inspected using a phase contrast microscope (Zeiss Axiovert 25, 400× magnification). The cell appearance was significantly improved when sufficient levels of the alpha-hydroxy acid derivatives were present in the medium. Only single cells were observed and no aggregation of cells was seen. The cell shape was also positively affected. Cells had a much more round and bright appearance when cultured in medium containing sufficient levels of the alpha-hydroxy acid derivatives. This was in contrast with the observation that a lot of cell aggregates were present in CHO cell cultures grown in chemically defined medium without alpha-hydroxy acid derivatives. The cell growth and production data are summarized in Table 2.

TABLE 2
Specific IgG production and cell growth of CHO cells in
chemically defined cell culture medium (see Example 5
for details) with added alpha-hydroxy acid derivatives of
the invention in varying concentrations. Production and
growth data in chemically defined cell culture medium
without added alpha-hydroxy acid derivatives, as well as
in chemically defined cell culture medium supplemented
with soy protein hydrolysate (0.4% w/v) and with fetal calf
serum (Gibco-Invitrogen; 5% v/v) are provided for comparison.

| | | Specific IgG | Cell growth |
|---|---|---|---|
| | Concentration | production | (area under curve) |
| Components added | (% w/v) | after 11 days | after 14 days |
| Methyl-L-3-phenyl | 1 × 10⁻¹ | 77.80 | 46.70 |
| lactate | | | |
| Mucic acid | 1 × 10⁻¹ | 84.61 | 48.29 |
| | 1 × 10⁻² | 90.27 | 49.64 |
| | 1 × 10⁻³ | 90.85 | 49.88 |
| None | N/A | 68.2 | 55.5 |
| Soy protein | N/A | 70.9 | 69.1 |
| hydrolysate | | | |
| (0.4% w/v) | | | |
| Fetal Calf Serum | N/A | 41.9 | 217.6 |
| (5% v/v) | | | |

1. A process of producing a culture medium for culturing eukaryotic cells comprising adding to conventional culture medium ingredients, either as a pure substance or as a concentrate, one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids and combinations thereof, as a growth-promoting and/or production-improving ingredient,
such that the final concentration in the medium is at least 0.001 mg/l per individual alpha-hydroxy acid, its ester or its salt.

2. The process according to claim 1, wherein the final concentration in the medium is at least 0.01 mg/l.

3. The process according to claim 1, wherein said C₂-C₆ alpha-hydroxy acids are selected from L-3-phenyl lactic acid and mucic acid.

4. The process according to claim 1, further comprising adding a plant protein hydrolysate to said culture medium.

5. The process according to claim 4, wherein said plant protein hydrolysate is a wheat, soy, cotton, or pea protein hydrolysate, or a mixture of two or more of these hydrolysates.

6. The process according to claim 1, further comprising adding one or more amino acid derivatives selected from γ-glutamyl amino acids, pyroglutamyl amino acids, glutamate-containing or proline-containing dipeptides, oxo-aminoacids, homo-amino acids, N-acetyl-amino-acids and glycyl-glycine; phenolic acid derivatives; linear C₂-C₆ or cyclic C₆ sugar alcohols; pyridinic acid derivatives; and nucleobases and/or nucleotides chosen from uracil, adenine, adenosine 3′-monophosphate (3′-AMP) and adenosine 5′-monophosphate (AMP), such that the final concentration in the medium is at least 0.001 mg/l.

7. (canceled)

8. A culture medium for culturing eukaryotic cells comprising at least 0.001 mg per l, or at least 0.02 mg per kg of dry matter, of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids and combinations thereof.

9. The culture medium according to claim 8, comprising at least 0.01 mg per l, or at least 0.2 mg per kg of dry matter of one or more C₂-C₆ alpha-hydroxy acids selected from L-3-phenyl lactic acid, mucic (galactaric) acid, gluconic acid, glucaric acid, glyceric acid, 2-hydroxy butyric acid, alpha-hydroxyisovaleric acid, alpha-hydroxyisocaproic acid and erythronic acid, salts of these acids, esters of these acids and combinations thereof.

10. The culture medium according to claim 8, comprising at most 50 g/l per said individual component, or at most 1000 g per kg of dry matter of one or more of said C₂-C₆ alpha-hydroxy acids, their esters, their salts, or combinations thereof, wherein said concentrations are per individual component.

11. The cell culture medium according to claim 8, comprising one or more of said C₂-C₆ alpha-hydroxy acids, salts of these acids, esters of these acids and combinations thereof in a concentration of between 5 mg/l and 30 g/l, or between 100 mg and 600 g per kg dry matter per individual component.

12. The culture medium according to claim 8, further comprising one or more conventional constituents of culture media selected from vitamins, minerals, amino acids, buffering salts, trace elements, nucleosides, nucleotides, phytohormones, sugars, antibiotics and protein hydrolysates.

13. The culture medium according to claim 8, further comprising one or more hydrolysates from wheat, soy, cotton, or pea protein.

14. The culture medium according to claim 8, further comprising one or more amino acid derivatives consisting of γ-glutamyl amino acids, pyroglutamyl amino acids, glutamate-containing or proline-containing dipeptides, oxo-aminoacids, homo-amino acids, N-acetyl-amino-acids and glycyl-glycine; phenolic acid derivatives; linear C₂-C₆ or cyclic C₆ sugar alcohols; pyridinic acid derivatives; and nucleobases and/or nucleotides chosen from uracil, adenine, adenosine 3′-monophosphate (3′-AMP) and adenosine 5′-monophosphate (AMP), or combinations of compounds from these compound groups.

15. The culture medium according to claim 8, wherein the amino acid derivatives are selected from γ-glutamyl-tyrosine, γ-glutamyl-phenylalanine, cyclo-glycyl-glutamate, valinyl glutamate, 5-oxoproline and β-alanine.

16. The culture medium according to claim 8, wherein the phenolic acids are chosen from ferulic acid ((E)-3-(4-hydroxy-3-methoxy-phenyl)prop-2-enoic acid), syringic acid (4-hydroxy-3,5-dimethoxybenzoic acid), vanillic acid (4-hydroxy-3-methoxybenzoic acid), sinapinic acid (3-(4-hydroxy-3,5-dimethoxyphenyl)prop-2-enoic acid), esters of these acids, or salts of these acids, preferably ferulic acid, syringic acid, esters of these acids, or salts of these acids, salts of these acids and esters of these acids.

17. The culture medium according to claim 8, comprising one or more compounds selected from chiro-inositol, erythritol, threitol, sorbitol and trigonelline.
